(19) United States
(12) Patent Application Publication
(10) Pub. No.: US 2021/0068637 A1
(43) Pub. Date: Mar. 11, 2021

(54) ENDOSCOPE AND CHANNEL TUBE

(71) Applicant: OLYMPUS CORPORATION, Tokyo (JP)

(72) Inventor: Eijiro SATO, Tokyo (JP)

(73) Assignee: OLYMPUS CORPORATION, Tokyo (JP)

(21) Appl. No.: 17/019,933

(22) Filed: Sep. 14, 2020

Related U.S. Application Data
(63) Continuation of application No. PCT/JP2018/029833, filed on Aug. 8, 2018.

(30) Foreign Application Priority Data
Mar. 16, 2018 (JP) 2018-049128

(51) Int. Cl. A61B 1/018; A61B 1/005; A61B 1/05

(52) U.S. Cl. CPC A61B 1/018; A61B 1/05; A61B 1/005

(57) ABSTRACT

An endoscope includes an insertion portion that is inserted into a subject, and a flexible channel tube that is disposed on an outer peripheral side of the insertion portion, includes a tube through-hole that is formed to be long along a tube center axis, and has a slit formed throughout an entire length, and the slit is formed in a section substantially orthogonal to the tube center axis of the tube through-hole of the channel tube with an extension line of a central line extending to a through-hole side of the slit central line of the slit being displaced with respect to the tube center axis of the channel tube.

### CROSS REFERENCE TO RELATED APPLICATION

This application is a continuation application of PCT/JP2018/029833 filed Aug. 8, 2018 and claims benefit of Japanese Application No. 2018-049128 filed in Japan on Mar. 16, 2018, the entire contents of which are incorporated herein by this reference.

### BACKGROUND OF THE INVENTION

### 1. Field of the Invention

The present invention relates to an endoscope with a channel tube for inserting a treatment instrument on an outer peripheral side of an insertion portion, and the channel tube that is used in the endoscope.

### 2. Description of the Related Art

In a medical field, medical endoscopes which can perform observation of organs in body cavities and the like, and various medical treatments by using treatment instruments by inserting elongated insertion portions into body cavities are widely used. An endoscope used in the medical field includes an elongated insertion portion, and a bending portion for orienting a position of a distal end portion in a desired direction is provided at a distal end side of the insertion portion.

A treatment instrument inserting channel is provided in the insertion portion. A surgeon can perform various medical treatments and the like by inserting a treatment instrument into the treatment instrument inserting channel as necessary during observation. For example, when the surgeon finds a polyp, a calculus or the like, the surgeon inserts a treatment instrument for treatment into the treatment instrument channel and resects of the polyp, crushes the calculus, or the like. Thereafter, the surgeon inserts a treatment instrument for collection into the treatment instrument channel in place of the treatment instrument for treatment, and extracts the treatment instrument for collection to collect the resected polyp, crushed calculus fragments, or the like.

More specifically, when the resected polyp is small, the surgeon performs collection by inserting the treatment instrument for collection into the treatment instrument channel, pinching the polyp and extracting the treatment instrument for collection. In contrast to this, when the resected polyp is large, the surgeon captures the polyp with the treatment instrument for collection that is inserted into the treatment instrument channel, and extracts the treatment instrument with the insertion portion from the inside of the body cavity while holding the capturing state to perform collection.

Here, when a plurality of resected polyps exist and the polyps are small, the surgeon performs collection by repeating insertion and extraction of the treatment instrument for collection into and from the treatment instrument channel.

When the existing resected plurality of polyps are large, the surgeon performs collection by repeating a procedure of introducing the insertion portion into the body cavity, a procedure of capturing the polyps with the treatment instrument fax collection, and a procedure of extracting the treatment instrument with the insertion portion from the inside of the body cavity. When a plurality of resected polyps exist, and the polyps are large, a burden on the doctor and the patient is large.

Japanese Patent Application Laid-Open Publication No. 2013-188638 discloses an auxiliary instrument for endoscope apparatus including an annular cap, and a treatment instrument insertion conduit along an outer peripheral side of the cap. In the treatment instrument insertion conduit of the auxiliary instrument for endoscope apparatus, a slit that allows an inside and an outside of the treatment instrument insertion conduit to communicate with each other is formed continuously from an outlet end to an inlet end side.

According to the configuration, the auxiliary instrument for endoscope apparatus can be used by being easily attached to a ready-made endoscope apparatus. The slit is continuously formed from the outlet end to the inlet end side, and therefore even with the endoscope apparatus to which the auxiliary instrument for endoscope apparatus is attached being inserted inside the body cavity, a net holding collected polyps and the like can be taken out of the body cavity along the slit formed in the treatment instrument insertion conduit by pulling a string connecting to the net holding the collected polyps and the like.

### SUMMARY OF THE INVENTION

An endoscope of one aspect of the present invention includes an insertion portion that is inserted into a subject, and a flexible channel tube that is disposed on an outer peripheral side of the insertion portion, includes a through-hole that is along a longitudinal axis, and has a slit formed throughout an entire length in the longitudinal axis direction, wherein a central line of the slit in a section substantially orthogonal to a center axis of the through-hole inclines with respect to a line segment connecting the center axis and the slit.

A flexible channel tube according to one aspect of the present invention is capable of being disposed on an outer peripheral side of an insertion portion that is inserted into a subject, and includes a through-hole that is along a longitudinal axis, and a slit throughout an entire length in the longitudinal axis direction, wherein a central line of the slit in a section substantially orthogonal to a center axis of the through-hole inclines with respect to a line segment connecting the center axis and the slit.

### BRIEF DESCRIPTION OF THE DRAWINGS

FIG. 1 is a view explaining an endoscope having an insertion portion, and including a channel tube disposed on an outer peripheral surface side of the insertion portion;

FIG. 2A is a view explaining a distal end surface of the insertion portion, which is a view of the distal end surface of the insertion portion seen in an arrow Y2A direction in FIG. 1;

FIG. 2B is a sectional view taken along a line Y2B-Y2B in FIG. 2A, which is a view explaining a relationship of the insertion portion and the channel tube;

FIG. 2C is a sectional view taken along a line Y2C-Y2C in FIG. 2B, which is a view explaining a slit of the channel tube;

FIG. 2D is a view explaining a relationship of a slit central line in the channel tube fixedly provided at a distal end of the insertion portion and a bending direction of a bending portion;

FIG. 3A is a view explaining fitting of a treatment instrument to the channel tube;

FIG. 3B is a view explaining a procedure of expanding a slit and fitting a treatment instrument insertion portion to a tube through-hole;

FIG. 3C is a view illustrating the treatment instrument insertion portion placed in the tube through-hole;

FIG. 3D is a view explaining and illustrating a state where the treatment instrument insertion portion having an outside diameter larger than an inside diameter of the tube through-hole is placed in the tube through-hole;

FIG. 4 is a view explaining a channel tube having a slit with a uniform width;

FIG. 5A is a view explaining a channel tube having a recessed portion in an outer shape;

FIG. 5B is a view explaining a relationship of the tube through-hole of the channel tube and the treatment instrument insertion portion;

FIG. 5C is a view explaining and illustrating a state where the treatment instrument insertion portion having the outside diameter larger than the inside diameter of the tube through-hole is placed inside the tube through-hole;

FIG. 6 is a view explaining another channel tube different in shape of a recessed portion;

FIG. 7A is a view explaining a channel tube with a distal end to a proximal end being fixedly provided at the distal end portion; and

FIG. 7B is a view explaining a channel tube in which tube end portion inclined surfaces are each provided at a tube distal end surface side and a tube proximal end surface.

### DETAILED DESCRIPTION OF THE PREFERRED EMBODIMENT

Hereinafter, an embodiment of the present invention will be described with reference to the drawings.

Note that in the respective drawings used in the following explanation, a scale may be different for each of the components to make the respective components have enough sizes to be recognizable on the drawings. In other words, the present invention is not limited to only the numbers and quantities of the components, the shapes of the components, the ratios of the sizes of the components, and relative positional relationships of the respective components described in the drawings.

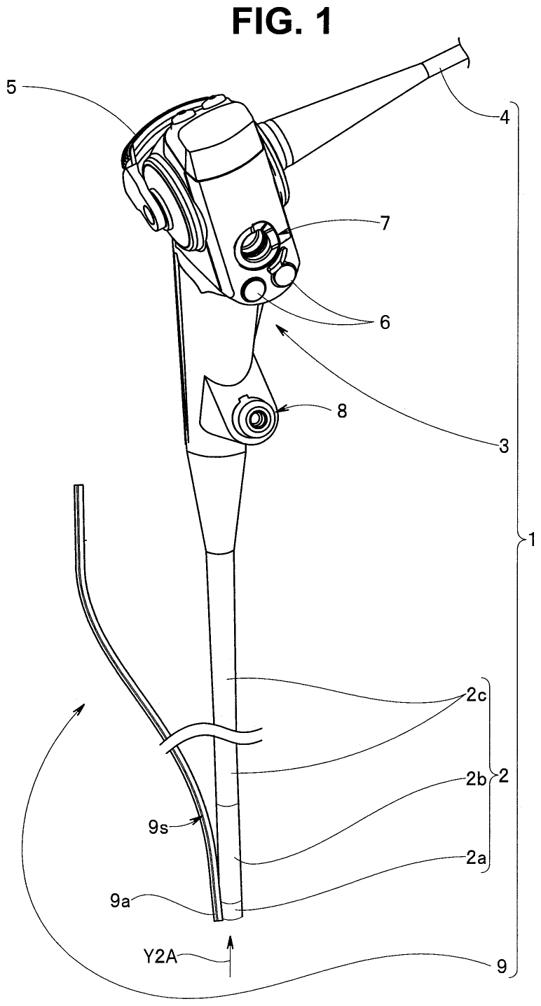

As illustrated in FIG. 1, an endoscope I has an insertion portion 2, an operation portion 3, a universal cable 4, and a channel tube 9. In other words, the endoscope 1 is an endoscope with a channel tube.

The insertion portion 2 is an elongated and long member that is inserted into a subject that is a site to be observed. The channel tube 9 is provided to be located on an outer side of an outer periphery of the insertion portion 2.

In the insertion portion 2, an insertion portion distal end portion (hereinafter, abbreviated as a distal end portion) 2a, a bending portion 2b, and a flexible tube portion 2c are provided continuously in order from a distal end side. The bending portion 2b is configured to be bendable in two directions, up and down, for example. The flexible tube portion 2c is a tubular member that is long and has flexibility.

Note that the bending portion 2b may be configured to be bendable in four directions, up, down, left, and right. The insertion portion 2 may be configured such that a rigid tube portion is continuously provided al a proximal end side of the bending portion 2b, instead of the flexible tube portion 2c.

In the operation portion 3, a bending operation portion 5, various switches 6, a cylinder 7, a treatment instrument insertion port 8 and the like are provided. The bending operation portion 5 is operated when causing the bending portion 2b to be operated to bend. The various switches 6 are, for example, a release switch, a freeze switch, or an observation mode change-over switch for performing switching of normal observation and fluorescence observation.

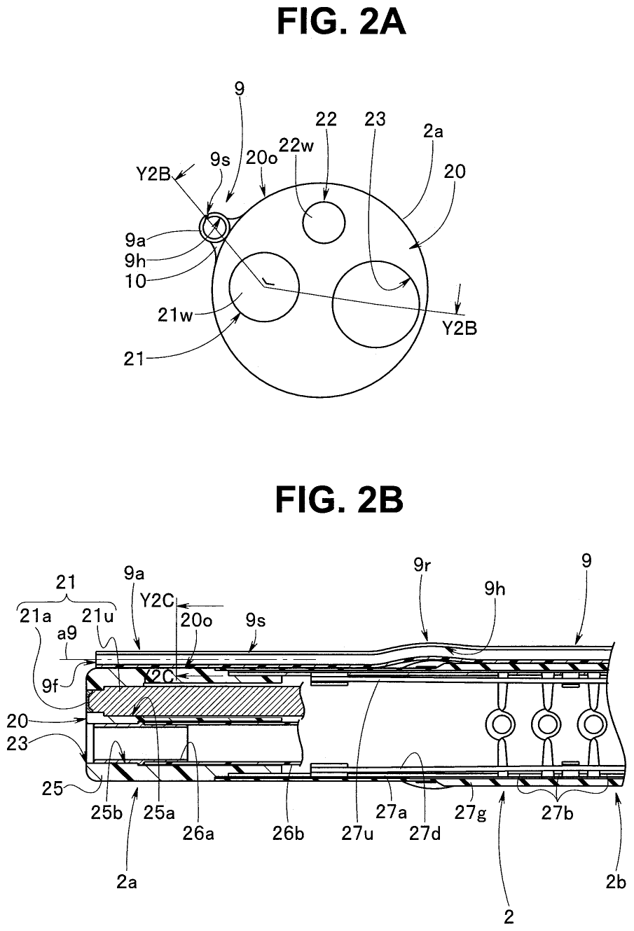

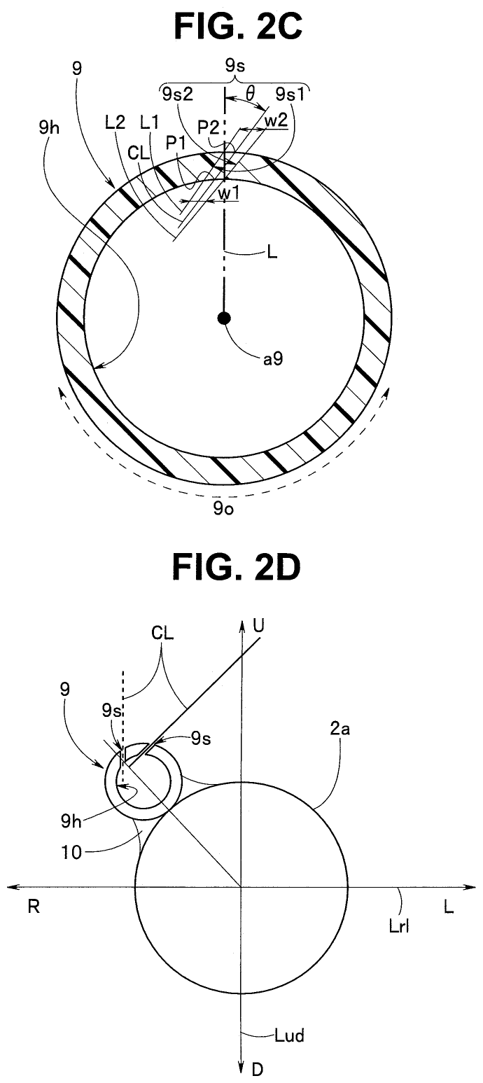

In the cylinder 7, a fluid control device (not illustrated) is disposed. The treatment instrument insertion port 8 communicates with an opening for treatment instrument channel (reference sign 23 in FIG. 2A) of the insertion portion distal end portion (hereinafter, abbreviated as the distal end portion) 2a via a treatment instrument inserting channel tube (refer to reference sign 26b in FIG. 2B). The treatment instrument inserting channel tube also serves as a channel for fluid for suction, water feeding and the like.

The universal cable 4 extends from a side portion of the operation portion 3. An endoscope connector not illustrated is provided at an end portion of the universal cable 4. The endoscope connector is attachable to and detachable from a camera control unit including, for example, a light source apparatus that is an external apparatus.

The channel tube 9 is made of a resin or a rubber having preset flexibility. The channel tube 9 has a tube distal end portion 9a fixedly provided at the distal end portion 2a.

A sectional shape of the channel tube 9 is substantially circular, and the channel tube 9 is formed to be long with a preset outside diameter. The channel tube 9 is equipped with a tube through-hole (reference sign 9h in FIG. 2B) along a tube center axis a9, and a slit 9s.

As illustrated in FIG. 2A, an observation window 21w of an image pickup optical system 21, and an illumination window 22w of an illumination optical system 22 are provided on a distal end surface 20 of the insertion portion 2. Reference sign 23 denotes the opening 23 for treatment instrument channel and also serves as an opening for fluid.

Reference sign 25 shown in FIG. 2B denotes a distal end rigid portion. The distal end rigid portion 25 configures the distal end portion 2a of the insertion portion 2. The distal end rigid portion 25 is provided with a through-hole 25a for observation optical system, a through-hole 25b for treatment instrument channel, a through-hole for illumination optical system (not illustrated) and the like.

As illustrated in FIG. 2A and FIG. 2B, in the through-hole 25a for observation optical system, the observation window 21w, and an image pickup unit 21u are placed. The observation window 21w configures an image pickup optical system, and the image pickup unit 21u has an image pickup member, an objective optical system and the like not illustrated. A distal end portion of a pipe sleeve 26a is fixedly provided in the through-hole 25b for treatment instrument channel. One end portion of the treatment instrument channel tube 26b is fixedly provided at a proximal end portion of the pipe sleeve 26a. The illumination window 22w, and LED lighting (not illustrated) are fixedly provided in the through-hole for illumination optical system.

In the present embodiment, the opening 23 for treatment instalment channel, the through-hole 25b for treatment instrument channel, the pipe sleeve 26a, and the treatment instrument channel tube 26b configure a treatment instrument inserting channel that also serves as the channel for fluid.

In the present embodiment, as illustrated in FIG. 2B, a distal end side of a distal end bending piece 27a configuring the bending portion 2b is integrally fixed to a proximal end side of the distal end rigid portion 25. Reference sign 27b denotes a plurality of bending pieces continuously provided at the distal end bending piece 27.

The bending portion 2b includes the bending pieces 27a and 27b, a bending rubber 27g covering the bending pieces 27a and 27b and the like. A distal end portion of an upper bending wire 27u and a distal end portion of a lower bending wire 27d are respectively fixed to preset positions of the distal end bending piece 27a.

Note that the endoscope may be a so-called rigid scope that includes no bending portion in the insertion portion.

As illustrated in FIG. 2A, in the channel tube 9, a tube distal end portion 9a that is one end portion of the channel tube 9 is fixedly provided at the distal end portion 2a. The tube distal end portion 9a is disposed on a distal end portion outer peripheral surface 20o that is an outer peripheral surface of the distal end portion 2a, and is integrally fixed to the distal end portion 2a by bonding, or welding.

As illustrated in FIG. 2B, a channel tube proximal end side portion (hereinafter, described as a tube proximal end side portion) 9r at a proximal end side from the tube distal end portion 9a of the channel tube 9 in the fixedly provided state is movable with respect to an outer periphery of the insertion portion 2. Reference sign 9f denotes a tube distal end surface, and the tube distal end surface 9f is disposed on a substantially same plane as the distal end surface 20 of the insertion portion 2.

As illustrated in FIG. 2A to FIG. 2C, the slit 9s is formed in the channel tube 9.

As illustrated in a sectional view orthogonal to the tube center axis a9 of the channel tube 9 in FIG. 2C, the slit 9s has a first slit formation surface 9s1, and a second slit formation surface 9s2. A first extension line L1 including the first slit formation surface 9s1, and a second extension line L2 including the second slit formation surface 9s2 are set to intersect a virtual line L.

The virtual line L is a straight line going toward the tube center axis a9 from outside of the tube.

In the present embodiment, the first extension line L1 and the second extension line L2 are formed to be displaced respectively so as not to intersect the tube center axis a9. An opening width (hereinafter, described as an inner opening width) at a tube inner surface side of the slit 9s is set at w1, and an opening width (hereinafter, described as an outer opening width) at a tube outer surface side is set at w2. Reference sign CL denotes a slit central line.

The slit central line CL is a line passing through a middle point P1 of the inner opening width w1 and a middle point P2 of the outer opening width w2, and is set to go from outside toward an inside of the tube through-hole 9h. The slit central line CL is set to intersect the virtual line L going from outside of the tube toward the tube center axis a9 at a preset angle θ.

The slit 9s is formed to be displaced so that the central line CL extending into the tube through-hole 9h does not intersect the tube center axis a9.

Note that the outer peripheral surface 9o located at an opposite side to the slit 9s with the tube center axis a9 between the slit 9s and the outer peripheral surface 9o is a disposition surface at a time of the channel tube 9 being fixedly provided at a preset position of the distal end portion outer peripheral surface 20o of the distal end portion 2a.

As illustrated in FIG. 2D, the channel tube 9 is bonded and fixed to the distal end portion 2a by applying an adhesive 10, for example. An operator fixedly provides the channel tube 9 so that the slit central line CL and a bending direction line Lud shown by arrows U and D are in a positional relationship where the slit central line CL and the bending direction line Lud intersect each other as shown by solid lines. In other words, the operator prevents the slit central line CL and the bending direction line Lud from being in a positional relationship in which the slit central line CL and the bending direction line Lud are substantially parallel with each other and fixes the channel tube 9.

Note that when bending directions of the bending portion are four directions, the channel tube 9 is fixed so that the slit central line CL and the up-down bending direction line Lud, and the slit central line CL and a left-right bending direction line Lrl are not in substantially parallel positional relationships.

In this way, by making the direction of bending and the inclination direction of the slit different directions, it becomes difficult for a treatment instrument in the tube through-hole 9h to come out of the through-hole 9h when the bending portion 2b bends.

In the slit 9s, by setting the outer opening width w2 to be wider than the inner opening width w1, it becomes easy to introduce the treatment instrument into the tube through-hole 9h. Besides, it becomes difficult for the treatment instrument placed in the tube through-hole 9h to come out of the opening on the tube inner surface side.

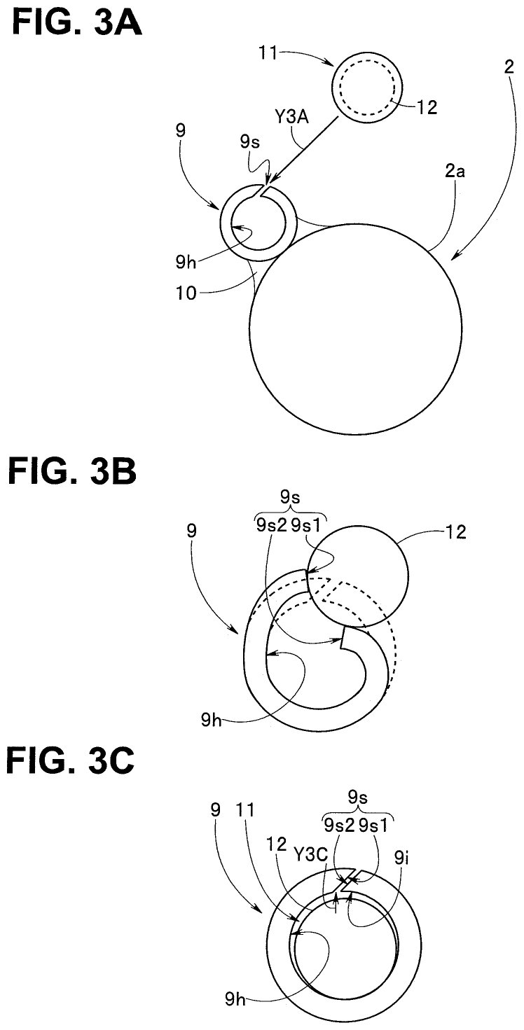

The endoscope 1 with the channel tube 9 disposed on the outer peripheral side of the insertion portion 2 will be described with reference to FIG. 3A to FIG. 3C.

In the endoscope 1 of the present embodiment with the channel tube 9 provided on the outer peripheral side of the insertion portion 2, it is possible to introduce treatment instruments in the channel tube 9 and the treatment instrument inserting channel tube provided in advance in the insertion portion 2, into a body cavity.

Therefore, a medical professional places a treatment instrument 11 in the channel tube 9 in advance. At this time, as illustrated in FIG. 3A, a treatment instrument insertion portion 12 of the treatment instrument 11 is caused to face the slit 9s leading to the tube through-hole 9h of the channel tube 9. Subsequently, the treatment instrument insertion portion 12 is moved toward the slit 9s as shown by an arrow Y3A.

The medical professional guides the treatment instrument insertion portion 12 into the tube through-hole 9h while elastically deforming the channel tube 9 so that the first slit formation surface 9s1 and the second slit formation surface 9s2 of the slit 9s open as illustrated in FIG. 3B. Subsequently, the treatment instrument insertion portion 12 is introduced into the tube through-hole 91a, and thereby the channel tube 9 that is elastically deformed returns to an original state.

By the above, as illustrated in FIG. 3C, the treatment instrument insertion portion 12 is placed inside the tube through-hole 9h, and fitting of the treatment instrument 11 into the channel tube 9 is completed.

Note that when an outside diameter of the treatment instrument 11 is smaller than an inside diameter of the tube through-hole 9h, the treatment instrument 11 may be fitted into the channel tube 9 by inserting a distal end portion of the treatment instrument 11 from a proximal end side opening of the channel tube 9 and inserting the distal end portion of the treatment instrument 11 toward a distal end side opening.

Here, the tube proximal end side portion 9r is movable to the outer peripheral surface of the insertion portion 2, and therefore, the treatment instrument insertion portion 12 that is placed inside the tube proximal end side portion 9r is also movable with respect to the outer peripheral surface of the insertion portion 2. Accordingly, when the insertion portion 2 is inserted toward a target site along a lumen that complicatedly bends, a position of the tube proximal end portion 13 in which the treatment instrument insertion portion 12 is placed freely changes correspondingly to a body cavity shape and an adverse influence on insertability is reduced.

In a treatment instrument fitted state illustrated in FIG. 3C, the first slit formation surface 9s1 configuring the slit 9s faces the second slit formation surface 9s2. The first slit formation surface 9s1 faces in a tube through-hole 9h direction, and the second slit formation surface 9s2 faces outward.

Therefore, when the insertion portion 2 is inserted toward the target site, the treatment instrument insertion portion 12 in the tube through-hole 9h may be moved in a slit 9s direction as shown by an arrow Y3, for example. At this time, the treatment instrument insertion portion 12 abuts on a tube inner peripheral surface 9i, and the second slit formation surface 9s2 abuts on the first slit formation surface 9s1. As a result, deformation of the second slit formation surface 9s2 is interfered with by the first slit formation surface 9s1 and the slit 9s is prevented from expanding.

Note that when the outside diameter of the treatment instrument insertion portion 12 is slightly larger than the inside diameter of the tube through-hole 9h, the distal end portion of the treatment instrument 11 may be inserted from the proximal end side opening of the channel tube 9, and may be inserted toward the distal end side opening while the slit 9s is elastically deformed to fit the treatment instrument 11 into the channel tube 9.

As illustrated in FIG. 3D, when the outside diameter of the treatment instrument insertion portion 12 is an outside diameter D2 shown by a solid line which is larger than an outside diameter D1 shown by a broken line, and is larger than the inside diameter of the tube through-hole 9h, the tube through-hole 9h is deformed to have a large diameter while the slit 9s is expanded. Thereby, the treatment instrument insertion portion 12 is placed inside the tube through-hole 9h.

When the first slit formation surface 9s1 and the second slit formation surface 9s2 are in a state where the first slit formation surface 9s1 and the second slit formation surface 9s2 face each other in the placement states, the second slit formation surface 9s2 abuts on the first slit formation surface 9s1 as described above, and the slit 9s is prevented from expanding.

In this way, the slit 9s in Which the slit central line CL is displaced with respect to the tube center axis a9 is provided in a side surface of the channel tube 9 throughout an entire length along the tube center axis a9 of the channel tube 9. Therefore, it is possible to place the treatment instrument insertion portion 12 in the tube through-hole 9h of the channel tube 9 by elastically deforming a vicinity of the slit 9s of the channel tube 9.

When the outside diameter of the treatment instrument insertion portion 12 is larger than the inner diameter of the tube through-hole 9h, it is possible to place the treatment instrument insertion portion 12 in the tube through-hole 9h of the channel tube 9 by expanding the slit 9s.

In addition, the channel tube 9 is provided on the insertion portion 2 with the positional relationship set such that the slit central line CL intersects the bending direction line Lud. Therefore, in the state where the treatment instrument insertion portion 12 is placed inside the tube through-hole 9h, the first slit formation surface 9s1 and the second slit formation surface 9s2 are in the positional relationship where the first slit formation surface 9s1 and the second slit formation surface 9s2 face each other.

Therefore, when the treatment instalment insertion portion 12 in the tube through-hole 9h is moved to a slit 9s side when the bending portion 2b is bent, or when the insertion portion 2 is inserted toward a target site, the second slit formation surface 9s2 abuts on the First slit formation surface 9s1 to prevent the treatment instrument insertion portion 12 from expanding the slit 9s, so that it makes hard for the treatment instrument to fall off outside through the slit 9s.

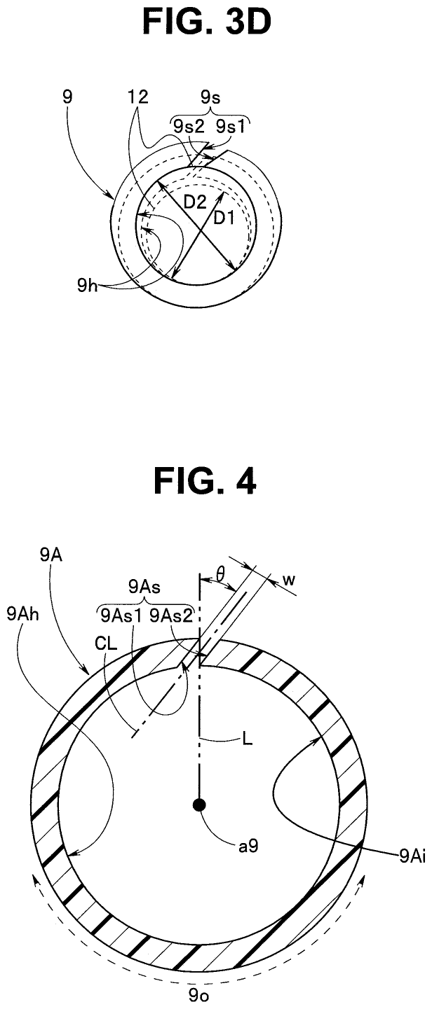

Another configuration example of the slit will be described with reference to FIG. 4.

As illustrating with a sectional view orthogonal to a tube center axis a9 of a channel tube 9A in FIG. 4, a slit 9As is formed in the channel tube 9A. The slit 9As of the present embodiment is set at a preset uniform width w. Accordingly, a slit central line CL divides the width w of the slit 9 into two. A first slit formation surface 9As1 and a second slit formation surface 9As2 are parallel surfaces.

In the present embodiment, the slit central line CL is also set to intersect a virtual line L going from outside of the tube toward the tube center axis a9 at a preset angle θ. The slit 9s is provided throughout an entire length of the channel tube 9A along the tube center axis a9 with the central line CL extending into the tube through-hole 9h displaced so as not to intersect the tube center axis a9.

The channel tube 9A is fixed to the distal end portion 2a as illustrated in FIG. 2D described above.

The other components are similar to the components of the aforementioned channel tube 9, and the same members are assigned with the same reference signs and explanation is omitted.

According to the channel tube 9A configured in this way, the first slit formation surface 9As1 and the second slit formation surface 9As2 face each other in parallel. Therefore, when the treatment instrument insertion portion 12 abuts on a tube inner peripheral surface 9i to deform the second slit formation surface 9As2 outward, the second slit formation surface 9As2 more reliably abuts on the first slit formation surface 9As1.

Accordingly, it is possible to make it more harder for the treatment instrument to fall off outside by effectively preventing the treatment instrument insertion portion 12 from expanding the slit 9As.

The other operations and effects are similar to the operations and effects of the aforementioned channel tube 9.

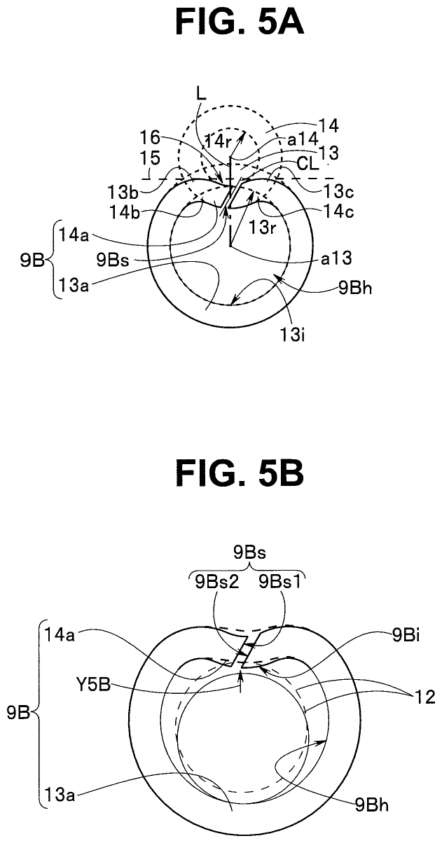

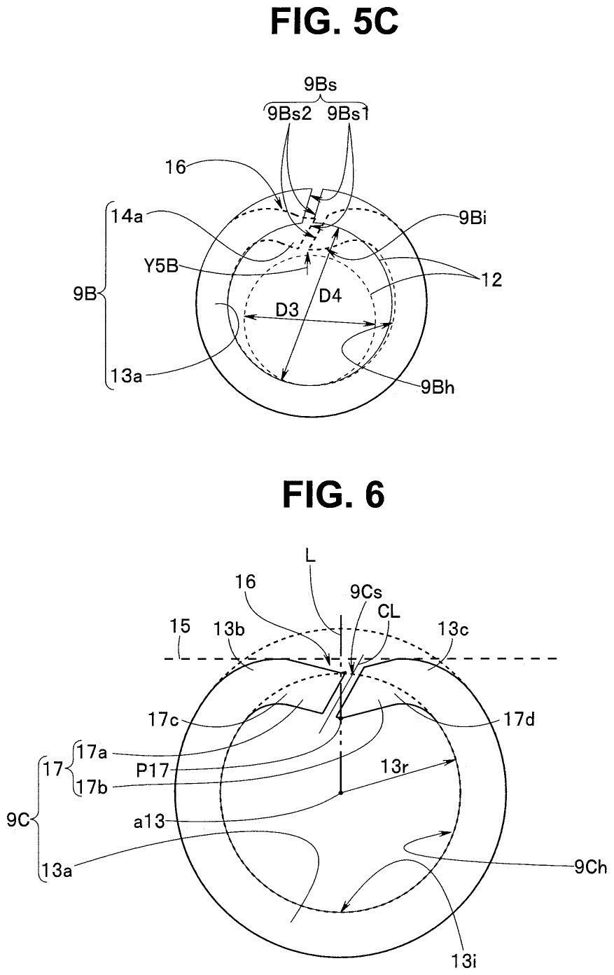

Note that in the channel tubes 9 and 9A described above, the tube sectional shapes are substantially circular. However, the sectional shape of the channel tube 9 is not limited to a circular shape, but as illustrated in FIG. 5A and FIG. 6, a first shape portion and a second shape portion may be included, and a recessed portion may be provided throughout the entire length of the channel tube 9.

With reference to FIG. 5A to FIG. 5C, another configuration example of the channel tube will be described.

A channel tube 9B illustrated in FIG. 5A is configured by a first circular arc 13a of a first circumferential portion 13, and a second circular arc 14a of a second circumferential portion 14. The first circular arc 13a is a first shape portion and is formed into a circular-arc shape, and the second circular arc 14a is a second shape portion forming a recessed portion 16 and is formed into a circular-arc shape.

Note that FIG. 5A is a sectional view orthogonal to a tube center axis a13 of the first circumferential portion 13 configuring the channel tube 9B, and a tube center axis a14 of the second circumferential portion 14.

In the present embodiment, the first circumferential portion 13 is formed with a first radius 13r, and the second circumferential portion 14 is formed with a second radius 14r smaller than the first radius 13r. The second circular arc 14a of the second circumferential portion 14 is disposed on an inner peripheral surface 13i side of the first circular arc 13a from a chord 15 located midway in the first circumferential portion 13.

Note that in the above, the second circumferential portion 14 is formed with the second radius 14r smaller than the first radius 13r. However, the radius of the second circumferential portion 14 may be set as same as the first radius 13r, or may be set as larger than the first radius 13r.

The channel tube 9B is formed into a heart shape by connecting a first end 14b of the second circular arc 14a and a first end 13b of the first circular arc 13a, and connecting a second end 14c of the second circular arc 14a and a second end 13c of the first circular arc 13a.

Note that the first end 13b of the first circular arc 13a and the second end 13c of the first circular arc 13a are portions at which the first circular arc 13a and the chord 15 intersect each other. The intersection portions are smooth curved surface portions as illustrated in FIG. 4A. Note that the intersection portions may be formed so that shapes abruptly change without being formed into the smooth curved surface portions.

In the channel tube 9B, the slit 913s is formed into the second circular arc 14a. The slit 9Bs includes either the slit 9s or the slit 9As shown in the aforementioned embodiments. The slit 9Bs in the present embodiment is set to have a uniform width w. The slit 9Bs is formed to be displaced so that a slit central line CL intersects a virtual line L that connects a tube center axis a13 of the first circumferential portion 13 and a center axis a14 of the second circumferential portion 14, but does not intersect the tube center axis a13.

The channel tube 9B is fixed to a distal end portion outer peripheral surface 20o of a distal end portion 2a so that the slit central line CL and the bending direction line Lud are in a positional relationship in which the slit central line CL and the bending direction line Lud intersect each other as illustrated in FIG. 2D described above.

As illustrated in FIG. 5B, a treatment instrument insertion portion 12 is placed inside a tube through-hole 9Bh of the channel tube 9B. In the present embodiment, the treatment instrument insertion portion 12 is also placed inside the tube through-hole 9Bh by expanding the slit 9Bs while elastically deforming a vicinity of the slit 9s.

Note that as described above, a treatment instrument 11 may be placed in the channel tube 9B by inserting a distal end portion of the treatment instrument 11 from a proximal end side opening of the channel tube 9B and inserting the distal end portion of the treatment instrument 11 toward a distal end side opening while elastically deforming the slit 9Bs.

When the treatment instrument insertion portion 12 which is placed inside tube through-hole 9Bh is moved in a slit 9Bs direction as illustrated by an arrow YSB, the treatment instrument insertion portion 12 abuts on a tube inner peripheral surface 9Bi.

Then, the second slit formation surface 9Bs2 abuts on the first slit formation surface 9Bs1 to deform a curvature of the second circular arc 14a gradually while preventing the slit 9Bs from expanding.

At this time, the second circular arc 14a changes from the recessed shape to a straight shape substantially corresponding to the chord 15, and to a protruded shape in which the second circular arc 14a is located at an outward side from the chord 15.

In the present embodiment, when an outside diameter of the treatment instrument insertion portion 12 of the treatment instrument 11 is an outside diameter D4 that is shown by a solid line and is a larger diameter than a D3 shown by a broken line and a larger diameter than an inside diameter of the tube through-hole 9Bh, the second circular arc 14a in the recessed shape changes into a protruded shape to place the treatment instrument insertion portion 12 inside the tube through-hole 9Bh. At this time, when the first slit formation surface 9Bs1 and the second slit formation surface 9Bs2 are in the state where the first slit formation surface 9Bs1 and the second slit formation surface 9Bs2 face each other, the slit 9Bs is prevented from expanding when the treatment instrument insertion portion 12 moves in the arrow Y5B direction.

In this way, the sectional shape of the channel tube 9B is formed into a shape in which the first circular arc 13a and the second circular arc 14a having the recessed portion 16 are combined from a circular shape, and the slit 9Bs is provided in the second circular arc 14a.

According to the configuration, in a time period until the second circular arc 14a forming the recessed portion 16 deforms into a substantially straight shape that substantially corresponds to the chord 15 while changing the curvature from the recessed shape, and in a time period until the second circular arc 14a deforms into a circular arc in the protruded shape from the straight shape, the second slit formation surface 9s2 and the first slit formation surface 9s1 move close to each other or abut on each other, and can make it hard for the treatment instrument insertion portion placed in the tube through-hole 9Bh to expand the slit 9s and fall off outside.

Note that the other operations and effects are similar to the operations and effects of the aforementioned embodiments.

Note that the aforementioned channel tube 9B has the first circular arc 13a, and the second circular arc 14a forming the recessed portion 16. However, the recessed portion of the channel tube is not limited to the recessed portion 16 provided in the second circular arc 14a, but may be a V groove recessed portion 17 illustrated in FIG. 6, or the like.

A channel tube 9C will be described with reference to FIG. 6.

As illustrated in FIG. 6, the channel tube 9C is formed of a first circular arc 13a that is a first shape portion, and the V groove recessed portion 17 that is a second shape portion. The present drawing is a sectional view orthogonal to a tube center axis a13 of a first circumferential portion 13 and a vertex P17 of the V groove recessed portion 17.

The V groove recessed portion 17 is a V groove including a first straight line portion 17a, a second straight line portion 17b, and the vertex P17. The vertex P17 of the V groove recessed portion 17 is disposed at an inner peripheral surface 13i side of the first circular arc 13a from a chord 15 as described above.

The channel tube 9C is formed into a substantially heart shape by connecting a first end 17c of the first straight line portion 17a and a first end 13b of the first circular arc 13a, and connecting a second end 17d of the second straight line portion 17b and a second end 13c of the first circular arc 13a. In other words, the channel tube 9C is configured by having the V groove recessed portion 17 including a recessed portion 16 in the present embodiment.

Similarly to the above, portions where the straight line portions 17a and 17b and the first circular arc 13a intersect one another are formed as smooth curved surface portions as illustrated in the drawing.

Note that the intersection portions may be formed such that shapes steeply change as described above.

In the channel tube 9C, the slit 9s is formed in the V groove recessed portion 17. The slit 9Cs includes either the slit 9s or the slit 9As shown in the aforementioned embodiments. The slit 9Cs of the present embodiment is set to have a uniform width w. The slit 9Cs is formed to be displaced so that a slit central line CL intersects a virtual line L connecting the tube center axis a13 of the first circumferential portion 13 and the vertex P17, but does not intersect the tube center axis a13.

Accordingly, the channel tube 9C is configured substantially similarly to the channel tube 9A, and includes similar operations and effects.

In addition, the channel tube 9C is fixed to a distal end portion outer peripheral surface 20o of a distal end portion 2a so that the slit central line CL and the bending direction line Lud are in a positional relationship where the slit central line CL and the bending direction line Lud intersect each other as illustrated in FIG. 2D described above.

According to the configuration, similarly to the aforementioned channel tube 9B, a treatment instrument insertion portion 12 (not illustrated in the present drawing) is placed inside the tube through-hole 9Ch by elastically deforming a vicinity of the slit 9Cs to expand the slit 9Cs.

Note that a treatment instrument 11 may be placed in the channel tube 9C by inserting a distal end portion of the treatment instrument 11 from a proximal end side opening of the channel tube 9C, and inserting the distal end portion of the treatment instrument 11 toward a distal end side opening while the slit 9Cs is elastically deformed.

When the treatment instrument insertion portion 12 that is placed inside the tube through-hole 9Ch is moved in a slit 9Cs direction, the V groove recessed portion 17 is deformed, thereafter, a second slit formation surface 9Cs2 abuts on a first slit formation surface 9Cs1 to be gradually deformed into a recessed shape, a straight shape, and a protruded shape, and the slit 9Cs is prevented from expanding.

When an outside diameter of the treatment instrument insertion portion 12 is a large diameter, the treatment instrument insertion portion 12 is placed inside the tube through-hole 9Ch by deforming the V groove recessed portion 17 from the V shape to an inverted V shape while expanding the slit 9Cs. When the first slit formation surface 9Cs1 and the second slit formation surface 9Cs2 are in the state where the first slit formation surface 9Cs1 and the second slit formation surface 9Cs2 face each other in the placement state, the treatment instrument insertion portion 12 is moved in a slit 9Cs direction, the second slit formation surface 9Cs2 also abuts on the first slit formation surface 9Cs1, and the slit 9Cs is prevented from expanding.

In this way, it is possible to obtain similar operations and effects to the operations and effects of the channel tube 9B by forming the sectional shape of the channel tube 9C into a substantially heart shape by combining the first circular arc 13a and the V groove recessed portion 17. The other operations and effects are similar to the operations and effects of the aforementioned embodiments.

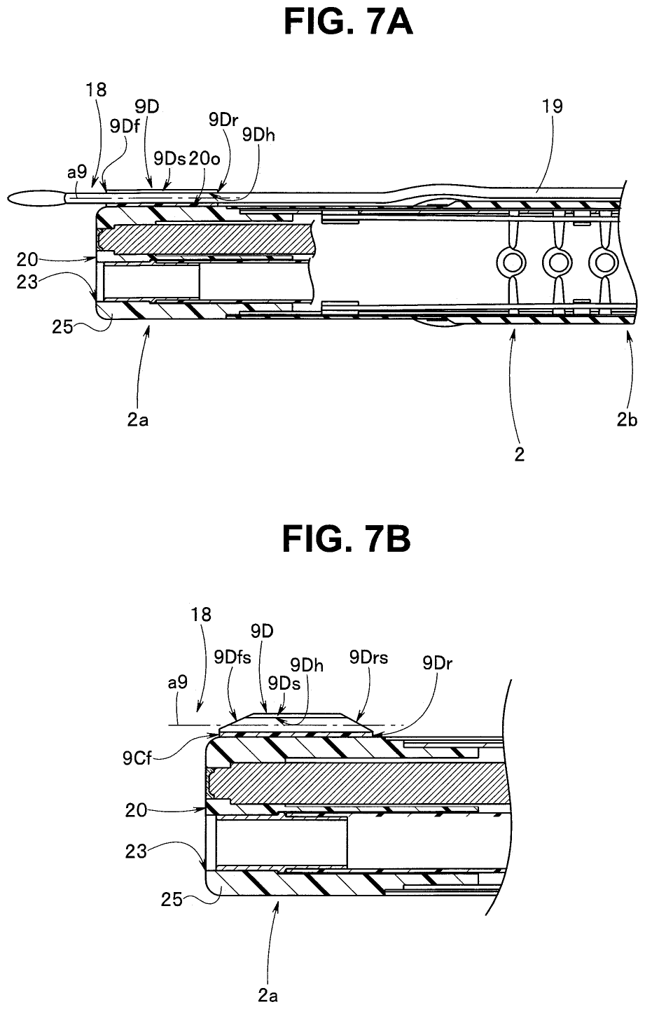

In the aforementioned embodiments, in each of the channel tubes 9, 9A, 9B, and 9C, the tube distal end portion 9a is fixedly provided at the distal end portion 2a, and the tube proximal end side portion 9r is movable with respect to the outer periphery of the insertion portion 2. However, a channel tube 9D may be configured as illustrated in FIG. 7A.

Still another configuration of the channel tube will be described with reference to FIG. 7A.

As illustrated in FIG. 7A, in the channel tube 9D of the present embodiment, a distal end to a proximal end are disposed on a distal end portion outer peripheral surface 20o of a distal end portion 2a and integrally fixed by bonding or welding. In the channel tube 9D of the present embodiment, a slit 9s is also provided as illustrated in FIG. 2C described above.

The channel tube 9D is fixed to the distal end portion outer peripheral surface 20o of the distal end portion 2a so that a slit central line CL and a bending direction line Lud are in a positional relationship in which the slit central line CL and the bending direction line Lud intersect each other as illustrated in FIG. 2D described above.

According to the configuration, from a tube proximal end surface 9Dr of the channel tube 9D, only a treatment instrument insertion portion 19 of a treatment instrument 18 is extended movably with respect to an outer peripheral surface of the insertion portion 2 without being covered with the channel tube 9D.

Therefore, the treatment instrument insertion portion 19 is disposed on an insertion portion outer peripheral surface at a proximal end side from the channel tube 9D. As a result, a sectional area as an endoscope system where the treatment instrument insertion portion 19 is disposed illustrated in FIG. 7B is smaller as compared with the sectional area as the endoscope system illustrated in FIG. 2B described above.

In addition, the treatment instrument insertion portion 19 is movable with respect to the insertion portion outer peripheral surface. Therefore, when the insertion portion 2 is inserted toward a target site along a lumen that complicatedly bends, the insertion portion 2 and the treatment instrument insertion portion 19 independently change freely to correspond to a body cavity shape and can reduce an adverse effect on insertability.

Note that as illustrated in FIG. 7B, a first tube end portion inclined surface 9Dfs is formed in a preset position on a tube distal end surface 9Df side of the channel tube 9D, and a second tube end portion inclined surface 9Drs is formed in a preset position on a tube proximal end surface 9Dr side of the channel tube 9D to form outside diameters on tube end surface sides into tapering shapes.

The first tube end portion inclined surface 9Dfs intersects a tube center axis a9 extended to a distal end side from the tube distal end surface 9Df at a preset angle. The second tube end portion inclined surface 9Drs intersects the tube center axis a9 extended to a proximal end side from the tube proximal end surface 9Dr at a preset angle.

As a result, the channel tube 9D is in a tapering shape in which an outer shape becomes smaller continuously toward the tube distal end surface 9Df side. In addition, the channel tube 9D is in a tapering shape in which the outer shape becomes smaller continuously toward the tube proximal end surface 9Dr side.

In this way, the tube end portion inclined surfaces 9Dfs and 9Drs are respectively provided at a distal end portion side and a proximal end portion side of the channel tube 9D that is fixedly provided at the distal end portion 2a of the insertion portion 2.

As a result, the distal end side of the distal end portion 2a on which the channel tube 9D having the tube end portion inclined surfaces 9Dfs and 9Drs is fixedly provided is formed to be tapered.

Therefore, it is possible to realize improvement in insertability of the insertion portion 2, in a single state of the endoscope 1 where the treatment instrument 18 is not placed in the channel tube 9D. At a time of extracting the insertion portion 2, it is possible to perform extraction of Me insertion portion 2 smoothly since the tube end portion inclined surface 9Drs is formed on the tube proximal end surface 9Dr side of the channel tube 9D that is fixedly provided on the distal end portion 2a. in other words, it is possible to more reduce an adverse effect on insertability of the insertion portion 2 where the channel tube 9D is provided on the distal end portion 2a.

Note that the present invention is not limited to only the embodiments described above, but can be carried out by being variously modified within the range without departing from the gist of the invention.

What is claimed is:

1. An endoscope comprising:
an insertion portion that is inserted into a subject; and
a flexible channel tube that is disposed on an outer peripheral side of the insertion portion, includes a through-hole that is along a longitudinal axis, and has a slit formed throughout an entire length in the longitudinal axis direction,
wherein a central line of the slit in a section substantially orthogonal to a center axis of the through-hole inclines with respect to a line segment connecting the center axis and the slit.

2. The endoscope according to claim 1,
wherein in the slit, a width of the slit is uniform in the section of the channel tube.

3. The endoscope according to claim 1,
wherein the channel tube includes, in the section
a first shape portion formed into a circular arc shape, and
a second shape portion that is disposed on an inner peripheral surface side of the first shape portion from a chord provided midway in the first shape portion to form a recessed portion, and
the slit is formed in the second shape portion.

4. The endoscope according to claim 3,
wherein in the channel tube, in the section,
the first shape portion is a first circular arc formed into a circular-arc shape with a first radius,
the second shape portion is a second circular arc that is disposed on an inner peripheral surface side of the first circular arc from the chord and is formed into a circular-arc shape with a same or different radius as or from the first radius, and
the slit is formed in the second circular arc.

5. The endoscope according to claim 3,
wherein in the channel tube, in the section,
the first shape portion is a first circular arc formed into a circular-arc shape with a first radius,
the second shape portion is a V groove recessed portion formed into a V shape having a vertex disposed on an inner peripheral surface side of the first circular arc from the chord, and
the slit is formed in the V groove recessed portion.

6. The endoscope according to claim 1, further comprising:
a bending portion that is provided in the insertion portion and is bendable by a bending operation device,
wherein the channel tube is disposed on an outer periphery of the insertion portion with an extending direction of an extension line of the slit and a bending direction of the bending portion being oriented in different directions in the section of the channel tube.

7. The endoscope according to claim 1, further comprising:
an image pickup member for observing the subject; and
an insertion portion distal end portion provided at a distal end portion of the insertion portion and configured to hold the image pickup member,
wherein a distal end to a proximal end of the channel tube is fixedly provided on an outer peripheral side of the insertion portion distal end portion.

8. The endoscope according to claim 7,
wherein a tube end portion inclined surface that makes a tube end surface side outer shape a tapering shape is provided at each of a preset position at a tube distal end side and a preset position at a tube proximal end side of the channel tube.

9. A flexible channel tube capable of being disposed on an outer peripheral side of an insertion portion that is inserted into a subject, comprising:
a through-hole that is along a longitudinal axis; and
a slit throughout an entire length in the longitudinal axis direction,
wherein a central line of the slit in a section substantially orthogonal to a center axis of the through-hole inclines with respect to a line segment connecting the center axis and the slit.

10. The channel tube according to claim 9,
wherein in the slit, a width of the slit is uniform in the section of the channel tube.

11. The channel tube according to claim 9, comprising:
a first shape portion formed into a circular arc shape; and
a second shape portion that is disposed on an inner peripheral surface side of the first shape portion from a chord provided midway in the first shape portion to form a recessed portion, in the section,
wherein the slit is formed in the second shape portion.

12. The channel tube according to claim 11,
wherein the first shape portion is a first circular arc formed into a circular-arc shape with a first radius,
the second shape portion is a second circular arc that is disposed on an inner peripheral surface side of the first circular arc from the chord and is formed into a circular-arc shape with a same or different radius as or from the first radius, and
the slit is formed in the second circular arc.

13. The channel tube according to claim 11,
wherein the first shape portion is a first circular arc formed into a circular-arc shape with a first radius,
the second shape portion is a V groove recessed portion formed into a V shape having a vertex disposed on an inner peripheral surface side of the first circular arc from the chord, and
the slit is formed in the V groove recessed portion.

14. The channel tube according to claim 9,
wherein a tube end portion inclined surface that makes a tube end surface side outer shape a tapering shape is provided at each of a preset position at a tube distal end side and a preset position at a tube proximal end side of the channel tube.
